(19) United States
(12) Patent Application Publication
(10) Pub. No.: US 2011/0269217 A1
(43) Pub. Date: Nov. 3, 2011

(54) HAZARDOUS SUBSTANCE ADSORBING TABLET

(75) Inventors: Heesoo EUN, Tsukuba-shi (JP); Hiroaki FUKUI, Kawaguchi-shi (JP)

(73) Assignees: Heesoo EUN, Tsukuba-shi (JP); TOKYO SINCOL CO., LTD., Tokyo (JP); NATIONAL INSTITUTE FOR AGRO-ENVIRONMENTAL SCIENCES, Tsukuba-shi (JP)

(21) Appl. No.: 13/079,466

(22) Filed: Apr. 4, 2011

Related U.S. Application Data
(63) Continuation-in-part of application No. PCT/JP2009/005213, filed on Oct. 7, 2009.

(30) Foreign Application Priority Data
Oct. 8, 2008 (JP) 2008-261258
Apr. 27, 2009 (JP) 2009-107293

(51) Int. Cl. C12N 1/20; C01B 31/08; B01J 29/06; B01D 39/00; B01J 20/24; B01J 20/02; B01J 20/26; B09C 1/08; B01J 20/00; B01J 21/16

(52) U.S. Cl. 435/253.6; 502/400; 502/409; 502/416; 502/60; 502/80; 502/404; 502/406; 502/412; 502/407; 502/402; 405/128.5; 71/64.13; 435/252.1; 210/502.1

(57) ABSTRACT

A hazardous substance adsorbing tablet prepared by direct compression of a dry powder mixture which includes a porous particulate adsorbent having an average particle diameter of 100 μm or less and containing 200 mesh pass particles in an amount of more than 80% by weight, and a particulate binder having an apparent specific gravity of at least 1.

### CROSS-REFERENCE TO RELATED APPLICATION

This application is a continuation-in-part of International Patent Application PCT/JP2009/005213, filed Oct. 7, 2009, which claims, under 35 USC 119, priority of Japanese Patent Applications No. 2008-261258 filed Oct. 8, 2008 and No. 2009-107293 filed Apr. 27, 2009, the entire contents of each of the above PCT and Japanese patent applications being hereby incorporated by reference herein in their entirety.

### BACKGROUND OF THE INVENTION

1. Field of the Invention

The present invention relates to a hazardous substance adsorbing tablet capable of adsorbing hazardous substances contained in soil or water in a polluted environment such as fields, paddy fields, rivers, groundwater, lakes, ponds, etc. The present invention is also directed to a method of treating a polluted environment using the above tablet.

2. Description of Prior Art

Powder spraying, such as agricultural chemical spraying or modifier spraying, is very hard work for elderly farmers. Also, it is difficult to uniformly spray a powdery material over the soil because of its high dusting property.

On the other hand, as a problem of soil contamination, it is known that hazardous substances, such as dioxins, prohibited agricultural chemicals, volatile organic substances and heavy metals, permeate the soil and are spread by water, such as rainwater or groundwater. Adsorption treatment methods for adsorbing the hazardous substances to solve the soil contamination problem include (a) topsoil replacement, (b) washing the soil with water and recovering the water used for the treatment by adsorption on an adsorbent such as activated carbon, (c) mixing microorganisms that decompose the hazardous substances into the soil, (d) injection of a redox agent into the ground to decompose the hazardous substances, and (e) soil incineration. However, in the case of the method (a), the hazardous substances may be carried by water again from untreated areas and cause recontamination without partitions or a water treatment facility. The method (b) is not suitable for an agricultural soil because it removes silt and rather destabilizes the land and because nutrients necessary for plants are also removed. In addition, the replacement, washing and decomposition detoxification of soil in the methods (a) (b) and (c) require a large amount of cost and a considerable processing time (several months to years).

Methods for reducing the hazardous substances in the soil or fixing the hazardous substances in the soil as countermeasures against soil contamination include (f) spraying a fine powder of an adsorbent, such as activated carbon, over the soil, (g) the use of cartridges filled with simple activated carbon, and (h) the use of an indisintegratable activated carbon sheet which contains activated carbon to adsorb the hazardous substances and is regenerated or disposed of by incineration after recovery. However, the method (f) is not suitable for use in the open air because the fine powder is difficult to distribute uniformly as it is easily blown up and scattered. One possible method to solve this problem is to build a simple plastic house over the soil to prevent the adsorbent powder from scattering. However, the construction of the simple plastic house rather increases the cost. Even if such a cover is provided, when the adsorbent is in the form of a fine powder consisting of fine particles with low specific gravity, it causes another problem: an increase in the risk of dust explosion in the simple greenhouse provided to prevent scattering of volatile components and dust. In addition, the method (f) requires a large amount of water because it takes a conventional way of spraying. Moreover, when water containing activated carbon hydrophilized with a surfactant is sprayed, the soil may bubble or contain a relatively excessive amount of water. Then, the soil turns into clay and makes it difficult for the workers to move to the next site of work. This places a burden on the workers. In this aging society, spraying of an agricultural chemical or modifier is very hard work for agriculture workers. The method (h), in which the soil is washed with water and the hazardous substances are recovered by adsorption on activated carbon, uses a large amount of drug to regenerate the activated carbon, and therefore has a high environmental load and requires much time and cost. In addition, because even components in the soil necessary for agricultural crops or living things may be removed, this method is not suitable as a soil reclamation method.

In addition, (I) drug tablets individually packaged in water soluble bags, and (J) foamable drug tablets are also used in special watery places such as paddy fields. For example, a sheet-like fertilizer prepared by impregnating a water soluble sheet with a fertilizer component or applying a fertilizer component to a water soluble sheet for easy and precise delivery of a proper amount of a component required by plants and for convenience in handling, and a throwing-in type agrochemical preparation composed of a water floatable solid agrochemical formulation containing a diffusing agent and wrapped in small portions with spreadable water soluble paper to disperse or dissolve active components in water are known. However, neither of them is suitable for soil treatment in a large area. The conventional sheet-like adsorbing formed body described above cannot treat a highly-contaminated areas into which a large amount of an adsorbent has been introduced. Also in this respect, the sheet-like adsorbing formed body still has a problem in use. Also, there has already been a method by which a finely pulverized product can be attached to a disintegrable or decomposable sheet but there has been no method by which a finely pulverized product can be uniformly applied to a sheet without deteriorating the performance of the finely pulverized product. For the above reasons, a simple and easy method by which recontamination can be prevented and hazardous substances can be fixed without large-scale equipment has been desired.

When a water soluble sheet is applied to a polluted water environment, such as rivers, lakes, ponds and groundwater, it is difficult to keep a water soluble sheet at the bottom of the river or the like due to its shape, and there is still a problem to be solved for uniform distribution of the hazardous substance adsorbent after the disintegration of the water soluble sheet unlike in the case where the polluted environment to which it is applied is a soil. It is even more difficult to apply a water soluble sheet to a flowing river, lake or pond. In addition, a method for adsorbing polluting substances spilled into groundwater or rivers or preventing diffusion of the polluting substances accumulated in the bottom sediment of the river or lake is highly desired but the conventional sheet-like adsorbing formed body is not sufficient to be applied to groundwater or river, or the bottom sediment of a river or lake in terms of its ability to adhere to the bottom of the river or lake. As an alternative to the sheet-like adsorbent described above, simple sprinkling of a fine powder of an adsorbent, such as activated carbon, to reduce or fix the hazardous substances in a water environment, such as a river, is considered. This method, however, has problems in use. Because the absorbent powder flies off, some dust control measure is required. Because the fine adsorbent powder consists of fine particles with low specific gravity, it floats on water and cannot stay at the site. Thus, even if a powdery hazardous substance adsorbent is applied to a polluted water environment, such as rivers, lakes, ponds and groundwater, the desired purpose cannot be achieved because the powder floats on water and cannot fulfill its function of adsorbing hazardous substances at the bottom. Another countermeasure against pollution in a water environment, such as rivers, is (J) a method to spread charcoal on the bottom of the river or lake. However, the method (J) is low in adsorption efficiency and requires a large amount of charcoal to be spread. In addition, the recovered soil needs a reclamation treatment.

In the field of pharmaceuticals, a tablet preparation method using a direct compression technique for preparing a disintegrating tablet with less weight variation, good disintegrability and practical hardness is known (JP-A-2007-197357). In this case, however, because the tablets are designed to disintegrate at the human body temperature according to Japanese Pharmacopoeia and are not intended to be applied to an open polluted environment, they do not disintegrate at a desired rate in cold water. Therefore, the tablets cannot have, nor are intended to have, controlled disintegratability in cold water at a temperature of 30° C. or lower in a water environment such as a river.

In the field of pharmaceutical preparation, it is also known to incorporate a finely pulverized product in a disintegrable or decomposable tablet. However, because the ingredients of the tablet are heated during the formation of the tablet, the intended performance of some of the ingredients are deteriorated. In addition, such a tablet does not disintegrate properly in a cold water at a temperature equal to or lower than human body temperature, and it is even more difficult to control the disintegration time of the tablet. One possible method to improve the disintegrability of the tablet is the use of a saccharide, such as lactose for direct compression tablets. Such a tablet, however, is not suitable for treatment of the water of a river or lake.

### SUMMARY OF THE INVENTION

It is, therefore, an object of the present invention to solve the problems of flying and floating of adsorbent powder that are caused when the absorbent is used to cope with environmental pollution in soil or water.

Another object of the present invention is to provide a tablet which can capture and adsorb hazardous substances contained in a polluted environment, so that the hazardous substances are confined therein and prevented from penetrating or diffusing into other places.

It is a further object of the present invention to provide a hazardous substance adsorbing tablet which has controlled disintegratability and can achieve the desired function of adsorbing hazardous substances. It is yet a further object of the present invention to provide a method which can effectively treat a polluted environment for adsorbing hazardous substances contained therein.

In accomplishing the foregoing objects, there is provided in accordance with the present invention a hazardous substance adsorbing tablet prepared by direct compression tableting of a dry powder mixture, the powder mixture including a porous particulate adsorbent having an average particle diameter of 100 μm or less and containing 200 mesh pass particles in an amount of more than 80% by weight, and a particulate binder having an apparent specific gravity of at least 1.

The above-described inventive tablet preferably disintegrates within 10 minutes when added into water at 4° C.

In any of the above-described inventive tablets, the porous particulate adsorbent is preferably at least one substance selected from the group consisting of calcite, naturally occurring zeolite, artificial zeolite, activated clay, diatomaceous earth, granite pegmatite, ilmenite and activated carbon having a specific surface area of 800 m²/g or more.

In any of the above-described inventive tablets, the porous particulate adsorbent and the particulate binder are present in amounts of 5 to 90% by weight and 10 to 95% by weight, respectively, wherein the total weight of the adsorbent and the binder is 100% by weight. In any of the above-described inventive tablets, the porous particulate adsorbent preferably has an average particle diameter of at least 1 μm.

In any of the above-described inventive tablets, the binder comprises at least one substance selected from the group consisting of crystalline cellulose, carboxymethyl cellulose, hydroxypropyl cellulose, starch, lactose, silicon dioxide, mannitol and anhydrous calcium hydrogen phosphate.

In any of the above-described inventive tablets, the binder preferably comprises 1 to 130 parts by weight of silicon dioxide and 100 parts by weight of crystalline cellulose.

In any of the above-described inventive tablets, the crystalline cellulose preferably has an average particle diameter 50 to 250 μm and silicon dioxide has an average particle diameter of 1 to 30 μm.

Any of the above-described inventive tablets preferably additionally contains at least one particulate adsorbing substance selected from the group consisting of inorganic salts which can form sparingly soluble heavy metal salts with heavy metal ions, inorganic sulfides which can form sparingly soluble heavy metal compounds with heavy metal ions, chelating agents, pH controlling agents, polyamino acids, polyacrylic acids and chelating agents.

Any of the above-described inventive tablets preferably additionally comprises at least one additive selected from the group consisting of oxidizing agents, reducing agents, microbial decomposing bacteria, slow oxygen-releasing agents, nutrients for growing bacteria, fertilizers, agricultural chemicals, and antibacterial antiseptic agents.

In another aspect, the present invention provides a method for treating a polluted environment, which includes applying any of the above-described inventive tablets to the polluted environment.

In the above inventive method, the polluted environment is preferably water or soil.

According to the hazardous substance adsorbing tablet of the present invention, there may be obtained the following effects. Namely, because of the particulate binder having an apparent specific gravity of at least 1 added to and compounded in the tablet, the tablet can sink at any desired speed when dropped into water. Further, the disintegration time of the tablet during the precipitation can be controlled.

As used herein, the term “apparent specific gravity” is intended to refer the apparent density of the sample at 4° C. and 1 atm divided by the apparent density of water at 4° C. and 1 atm.

In the case of tablets, application and treatment works may be very easily carried out by throwing, etc., irrespective of whether the applied place is water or soil. In the case of the treatment of a polluted environment such as river and groundwater, escape of fine powder by floating can be prevented. Further, it is possible to control dispersibility and buoyancy of the absorbent. Furthermore, it is possible to prevent flying and scattering of the fine powder and dust explosion and to improve the safety of workers and neighborhood. Additionally, the tablets may be produced at a relatively low cost, because the raw materials including the particulate binder having a specific gravity of at least 1 and porous particulate adsorbent may be pressed and tableted at a low pressure using a general tableting device.

Further, a tablet making process employed in the present invention is easier than a granule making process and can be performed in a short production time. Additionally, it is possible to freely select weight and size of the tablets according to object of use. Because the raw materials for the tablets can be, if desired, cellulose-based materials which have low environmental load, minerals which are stable in nature, and carbon, it is possible to design tablets in which safety is sufficiently taken into consideration.

It is also possible to effectively disperse fine powder adsorbent into the arid zone soil with the aid of small amount of water such as rain water, to allow the adsorbent to be suitably bound to the soil by stirring the applied soil and to prevent the adsorbent from flying and scattering. Further, hazardous substances can be fixed to the adsorbent so that the plants are prevented from absorbing the hazardous substances and the ground water is prevented from being polluted.

The tablet according to the present invention shows accelerated disintegratability even when a disintegrator is not incorporated. The reason for this is considered to be that the hardness of outer surfaces of the porous particulate adsorbent is increased during the course of tableting so that the air in interstices or large holes thereof is confined and compressed therein. As a result, when the tablet is exposed to a water-rich environment during use, a large amount of water is absorbed in the interstices or large holes by the capillary action with the simultaneous discharge of the confined air, so that disintegration of the tablet occurs at a burst.

It is inferred that, in the above phenomenon, the air discharged from the interstices or large holes of the porous powder serves to function as a foaming agent. For this reason, the tablet of the present invention prepared by a direct compression tableting method is considered to show accelerated disintegratability upon absorption of water. Although the tablet of the present invention is disintegratable without a disintegrator, a disintegrator may be incorporated into the tablet, if desired, to further accelerate the disintegration. Whether to incorporate a disintegrator may be determined at will. It has been found that when a tablet is prepared using a granulation device other than a compression tableting device, acceleration of disintegration is not achieved.

### BRIEF DESCRIPTION OF DRAWINGS

Other objects, features and advantages of the present invention will become apparent from the detailed description of the preferred embodiments of the invention which follows, when considered in light of the accompanying drawings in which:

FIG. 1 is a photograph showing the state of a tablet (prepared in Example 2) 3 seconds after having been dropped in water;

FIG. 2 is a photograph showing the state of a tablet (prepared in Example 3) 3 seconds after having been dropped in water;

FIG. 3 is a photograph showing the results of the test of dispersibility of a tablet of Example 7 in soil by water drops, and being a photograph showing the dispersing state of the activated carbon tablet 30 seconds after completion of dropping of water;

FIG. 4 shows the state of a tablet of Comparative Example 3, formed only of crystalline cellulose and silicon dioxide, 600 seconds after having been dropped in water at 15° C.;

FIG. 5 shows the state of a tablet prepared in Comparative Example 4, 600 seconds after having been dropped in water at 15° C.; and

FIG. 6 is a graph showing the results of the treatment of sludge in terms of change of α-HCH concentration with time.

### DETAILED DESCRIPTION OF THE PREFERRED EMBODIMENTS

The hazardous substance adsorbing tablet of the present invention is prepared by direct compression of a dry powder mixture containing (a) a porous particulate adsorbent having an average particle diameter of 100 μm or less and containing 200 mesh pass particles in an amount of more than 80% by weight, and (b) a particulate binder having an apparent specific gravity of at least 1. The content of the porous particulate adsorbent (a) is preferably 5 to 90% by weight, more preferably 10 to 80% by weight, still more preferably 10 to 50% by weight, based on the total weight of the adsorbent (a) and the binder (b), for reasons of adsorption efficiency and controllability of the disintegration rate and buoyancy.

In the present invention, one or a plurality of porous inorganic minerals and naturally occurring minerals such as calcite, zeolite, active clay, granite pegmatite and ilmenite or one or a plurality of artificial zeolite substances whose pore size is controlled, may be used as the porous particulate adsorbent. Further, activated carbon having a specific surface area of 800 m²/g or more is also preferably used by itself or in combination of the above porous minerals or artificial zeolite. Examples of artificial zeolite include Zeolite A, X or Y, ZSM zeolite and beta zeolite. Examples of naturally occurring zeolite include erionite, mordenite, clinoptilolite, chabazite, gmelinite, heulandite and faujasite. The natural and artificial zeolite may be exchanged with any suitable metal ions such as potassium ions and alkali earth ions.

If desired, the porous particulate adsorbent may be used together with one or more additional particulate adsorbing substances such as inorganic salts (e.g. hydroxide salts, carbonate salts, hydrogen carbonate salts, phosphate salts and sulfates) which can form sparingly soluble heavy metal salts with heavy metal ions, inorganic sulfides which can form sparingly soluble heavy metal compounds with heavy metal ions, chelating agents, pH controlling agents, polyamino acids (e.g. polyglutamic acid), polyacrylic acids and chelating agents. The polyamino acids which have fast-acting heavy metal adsorbing performance or water holding property and are capable of acting as a fertilizer after having been decomposed in the soil may be suitably used. The amount of the additional particulate adsorbing substance is generally not more than 50% by weight of the total amount of the porous particulate adsorbent and the additional particulate adsorbing substance.

It is important that the porous particulate adsorbent contain 200 mesh pass particles in an amount of more than 80% by weight in order to achieve the desired function of adsorbing hazardous substances. It is preferred that the porous particulate adsorbent have an average particle diameter of 1 to 100 μm, more preferably 10 to 100 μm, for reasons of adsorbing performance and tablet forming efficiency. The additional particulate adsorbing substance is also desired to contain 200 mesh pass particles in an amount of more than 80% by weight have an average particle diameter of 1 to 100 μm, more preferably 10 to 100 μm. The porous particulate adsorbent generally has an apparent specific gravity of less than 1.

The particulate binder having an apparent specific gravity of at least 1 may be, for example, crystalline cellulose, carboxymethyl cellulose, hydroxypropyl cellulose, starch, lactose, silicon dioxide, mannitol, anhydrous calcium hydrogen phosphate or a mixture of one or more thereof. The particulate binder generally has an average particle diameter of 1 to 1,000 μm. When two or more substances are used in combination as the particulate binder, it is not necessary that each of the substances should have an apparent specific gravity of at least 1. As long as the apparent specific gravity of the mixture of the substances is at least 1, one or more substances constituting the mixture may have an apparent specific gravity of less than 1.

For reasons of significantly improved disintegration controllability, low environmental load and harmlessness, it is particularly preferred that a mixture of crystalline cellulose and silicon oxide be used as the particulate binder. In this case, it is also preferred that silicon dioxide be used in an amount of 1 to 130 parts by weight, more preferably 2 to 100 parts by weight, still more preferably 2 to 50 parts by weight, per 100 parts by weight of crystalline cellulose. The crystalline cellulose preferably has an average particle diameter 50 to 250 μm and silicon dioxide preferably has an average particle diameter of 1 to 30 μm. When crystalline cellulose is used together with silicon dioxide, the crystalline cellulose having an apparent specific gravity of less than 1 may be used, if desired.

The tablet of the present invention may contain one or more additives such as an oxidizing agent, a reducing agent, microbial decomposing bacteria, a slow oxygen-releasing agent, a nutrient for growing bacteria, a fertilizer, an agricultural chemical and an antibacterial antiseptic agent. Further, plant essential amino acids, a nitrogen component, a phosphorus component, a potassium component and a foaming agent obtained by mixing an additive of a carbonate salt (e.g. potassium carbonate) or a hydrogen carbonate salt with a solid acid such as citric acid or ascorbic acid may be also used as the additive, if desired. The additive is generally used in an amount of 20% by weight or less, preferably 10% by weight or less, based on the weight of the tablet.

The size, proportion of the ingredients, disintegration speed, etc. of the tablet may be freely designed in view of the shape thereof and place of use thereof. Further, since the tablet may be produced by tableting with a low pressure, there is no need to use a specifically designed tableting device. A rotary tableting press machine used in food and pharmaceutical industries may be utilized for preparing the tablet of the present invention from a dry powder mixture of the above-described porous adsorbent, binder and optional ingredients.

In use of the molded tablets, application and treatment works may be very easily carried out by throwing, etc., irrespective of whether the applied place is water or soil. In the case of the treatment of river and groundwater, escape of fine powder by floating can be prevented. Further, it is possible to control disintegratability and buoyancy of the tablet and to increase the degree of freedom of adsorption performance of the tablet. The adsorbent may be composed of one or a plurality of porous particulate adsorbent with or without using the additional adsorbent material so that the adsorbing performance may be freely determined in consideration of the degree of pollution. The tablet may be formed into a single layer or multilayer structure by a direct compression tableting process. The ingredients of the tablet may be suitably selected to provide tablets having various disintegration times or tablets containing adsorbents having different performances.

The kind and concentration of pollutants vary with place to place. However, when the present invention is embodied as hazardous substance adsorbing tablets containing substances capable of adsorbing different kinds of organic hazardous compounds and inorganic hazardous compounds, it is possible to select adsorbents suited for adsorbing and fixing target pollutants. In particular, it is possible to freely change or design the performance of adsorbing hazardous components in correspondence to the highly polluted conditions.

The tablet of the present invention may contain an oxidizing agent, a reducing agent or a combination thereof, or organic substance decomposing bacteria since the bacteria are not damaged during low pressure compression tableting. By this expedient, it is possible to achieve bioremediation and to enhance decomposition of organic substances. Thus, the present invention can provide hazardous substance adsorbing tablets which can adsorb hazardous components in the soil when laid on surfaces of the soil or placed within the soil and when dissolved therein by sprinkling, rain, snow or moisture contained therein, which can be used in arid zone soil or for agriculture and which are high in safety.

As described previously, it is preferable to use a blend of crystalline cellulose (hereinafter referred to as cellulose) with silicon dioxide (hereinafter referred to as silica) as the particulate binder having an apparent specific gravity of at least 1. In this case, it is particularly preferred that the blended binder be used in conjunction with activated carbon (hereinafter referred to as carbon) having an average particle size of 100 μm or less and a specific surface area of 800 m²/g or more as the porous particulate adsorbent for the following reason.

That is, carbon cannot be tableted by itself. While a powder mixture of carbon with cellulose may be tableted by direct compression tableting, disintegratability of the obtained tablet cannot be controlled over a wide range. Similarly, while a tablet may be prepared from a powder mixture of carbon with silica by direct compression tableting, disintegratability of the obtained tablet cannot be controlled over a wide range. When cellulose is used in conjunction with silica as a particulate binder, on the other hand, there is unexpectedly obtained a synergetic effect that disintegration time and buoyancy of the resulting tablet can be controlled over a wide range at will. Although not wishing to be bound by the theory, it is inferred that the synergetic effect is obtained for the following reasons. When cellulose is used by itself as the binder, the cellulose shows hydrophobicity as a result of interaction between cellulose particles. Because carbon is also hydrophobic in nature, the tablet obtained therefrom tends to refuse intrusion of water so that the disintegratability is not high. When silica is used by itself as the binder, large holes of each carbon particle is filled with a large amount of silica so that the disintegratability is not high. When silica and cellulose are used conjointly, on the other hand, silica deposits on cellulose so that the cellulose exhibits hydrophilicity. The cellulose to which silica deposits enters into large holes of relatively large carbon particles, so that the carbon particles are in a state as if they were treated with a surfactant. In this case, carbon particles with small sizes also enter into the large holes. Upon compression tableting, air in the holes are confined therein. Further, cracks are formed in the composites of the carbon and silica/cellulose. When the tablet is contacted with water, water enters into the tablet through the cracks. Since the bonds between silica and cellulose and between carbon and silica/cellulose are by physical bonding, they are broken by intruded water to cause disintegration of the tablet.

### EXAMPLES

The following examples and comparative examples will further illustrate the present invention. Parts and percentages are by weight.

### Examples 1 to 6

Naturally occurring zeolite with an average particle diameter of 20 μm was used as a porous particulate adsorbent. Activated carbon having a specific surface area of 1,000 m²/g and an average particle diameter of 30 μm was used as a porous particulate adsorbent in Examples 2 to 6. As shown in Tables 1-1 and 1-2, in Examples 1 to 3 and 5 and 6, a powder mixture composed of 98% of crystalline cellulose and 2% of silicon dioxide was used as a particulate binder having an apparent specific gravity of at least 1. A powder mixture composed of 88% of crystalline cellulose, 10% of lactose and 2% of silicon dioxide was used as a particulate binder in Example 6. In Example 6, a foaming agent (combination of a metal carbonate and an organic acid) was also used. Tablets each having a diameter of 10 mm and a weight of 0.4 g were prepared from the dry powder mixtures having the compositions shown in Tables 1-1 and 1-2 at compression pressures shown in Tables 1-1 and 1-2 using a commercially available direct compression tableting machine. The obtained tablets of Examples 1 to 6 were tested for disintegration time in water and in soil. The results are summarized in Tables 1-1 and 1-2.

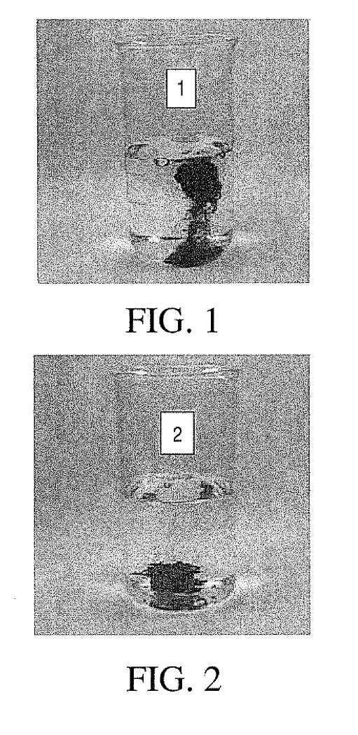

The tablets of Examples 1 and 2, when applied with water, disintegrated well on the soil. When added into water, they once sank in water, then floated and finally disintegrated. The state of disintegration of the tablet of Example 2 (3 seconds after dropping into water) is shown in FIG. 1. Thus, the produced tablets may be used for both land and water. Further, because the tablets of Examples 1 and 2 were prepared without using any foaming agent, a long term storage stability was ensured.

The tablets of Examples 3 and 4 sank in water and gradually disintegrated. FIG. 2 shows the state of the tablet (prepared in Example 3) 3 seconds after dropping into water. The test results show that it is possible to permit the tablets to disintegrate with a longer disintegration time in water, by decreasing the amount of the adsorbent (Example 3) or by additionally using a further vehicle as a part of the particulate binder (Example 4). That is, it can be said that the disintegration time is controllable. In Example 5, the tablet disintegrated and precipitated immediately after having been added into water. When the content of the adsorbent was high, an increase of the proportion of silicon dioxide permitted to obtain tablets that had fast disintegratability. It would be surprising that the tablet of the present invention can immediately disintegrate while generating air bubbles, even though no foaming agent is used. In Example 6, a foaming agent is additionally incorporated in an amount of 10%. The tablet disintegrated and precipitated immediately after having been added into water as in the case of Example 5. However, the tablet of Example 6 has a problem in storage stability because the foaming agent gradually reacts by contact with moisture and the foaming power is lost during storage. When the storage time is 6 months or less, the tablet can show fast disintegratability.

TABLE 1-1

| Example No. | 1 | 2 | 3 |
|---|---|---|---|
| Porous Adsorbent | Zeolite | Activated carbon | Activated carbon |
| Amount of adsorbent (g) | 0.08 | 0.08 | 0.04 |
| Binder | Mixture of crystalline | Mixture of crystalline | Mixture of crystalline |
| | cellulose (98%) and | cellulose (98%) and | cellulose (98%) and |
| | silica (2%) | silica (2%) | silica (2%) |
| Amount of binder (g) | 0.32 | 0.32 | 0.36 |
| Additive | — | — | — |
| Compression tableting | 1 to 2 | 3 | 1 to 2 |
| pressure (kN) | | | |
| Weight of tablet (g) | 0.4 | 0.4 | 0.4 |
| Diameter of tablet (mm) | 10 | 10 | 10 |
| Hardness (N) | 18 | 18 | over 20 |
| Distributed over soil | Disintegrated by water | Disintegrated by water | Disintegrated by water |
| Disintegration time in soil | less than 30 sec. | less than 30 sec. | less than 120 sec. |
| Distributed on water | Tablet sank in water, | Tablet sank in water, | Tablet sank in water and |
| | foamed and again floated | foamed and again floated | gradually disintegrated |
| Disintegration time in water | less than 10 sec. | less than 10 sec. | less than 60 sec. |

TABLE 1-2

| Example | 4 | 5 | 6 |
|---|---|---|---|
| Porous Adsorbent | Activated carbon | Activated carbon | Activated carbon |
| Amount of adsorbent (g) | 0.08 | 0.2 | 0.08 |
| Binder | Mixture of lactose (10%), | Mixture of crystalline | Mixture of crystalline |
| | crystalline cellulose (88%) | cellulose (98%) and | cellulose (98%) and |
| | and silica (2%) | silica (2%) | silica (2%) |
| Amount of binder (g) | 0.32 | 0.2 | 0.288 |
| Additive | — | — | foaming agent (0.032 g) |
| Compression tableting | 3 | 1 to 2 | 10 |
| pressure (kN) | | | |
| Weight of tablet (g) | 0.4 | 0.4 | 0.4 |
| Diameter of tablet (mm) | 10 | 10 | 10 |
| Hardness (N) | 15 | over 20 | 10 |
| Distributed over soil | Disintegrated by water | Disintegrated by water | Disintegrated by water |
| Disintegration time in soil | less than 120 sec. | less than 10 sec. | less than 30 sec. |
| Distributed on water | Tablet sank in water, | Disintegrated immediately | Disintegrated immediately |
| | and gradually disintegrated | after dropping, then | after dropping, and then |
| | | dispersed and precipitated | precipitated |
| Disintegration time in water | less than 60 sec. | less than 3 sec. | less than 10 sec. |

### Comparative Examples 1 to 4

Tablets were prepared under the conditions shown in Table 2. Comparative Example 1 used commercially available activated carbon fine powder having a specific surface area of 1,000 m²/g and an average particle diameter of 30 μm. The activated carbon fine powder was tried to be tableted by itself by compression tableting but was unable to produce tablets even with the maximum compression pressure of the tableting machine. It was impossible to form tablets when activated carbon fine powder was used by itself. Comparative Example 2 used similar activated carbon fine powder as such. In application to the soil, the fine powder scattered and formed a cloud of dust. In application to water, because of its small apparent specific gravity, the fine powder was afloat on the water surface. The activated carbon fine powder as such was found to be unsuited for application to polluted ground or water.

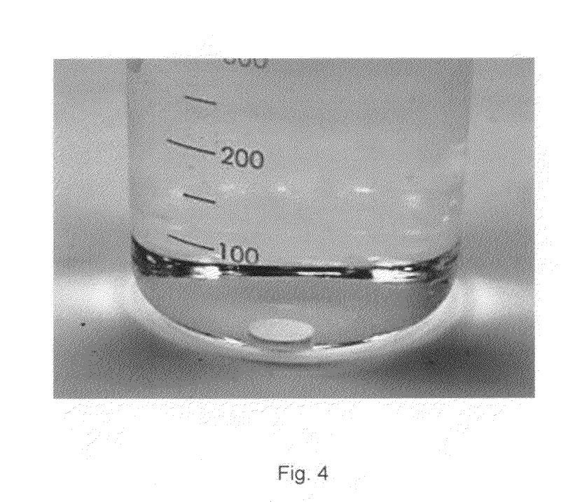

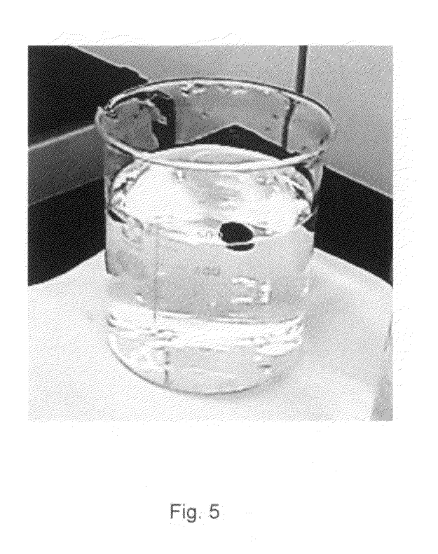

The tablet of Comparative Example 3 was prepared by compression tableting of a powder mixture composed of 98% of crystalline cellulose and 2% of silicon dioxide. No porous particulate adsorbent was used. Test results indicated that the tablet had good moldability. When applied to the soil, the tablet hardly disintegrated by water sprinkling. As shown in FIG. 4, the tablet sank in water but did not disintegrate when left in water at 15° C. for 600 seconds. Namely, the disintegratability of the tablet in an environment of low temperature water was very low. The tablet of Comparative Example 4 was prepared by compacting a mixture containing 5 parts of the above-described activated carbon fine powder with 1 part of water soluble polyvinyl alcohol. The resulting tablet when applied to the soil did not disintegrate by water sprinkling. FIG. 5 shows the state of the tablet 600 seconds after having been dropped in water at 15° C. As shown, the tablet floated on water and did not disintegrate. The tablet did not disintegrate even after one day. The tablet was found to be ill-suited for application to water of a polluted site.

TABLE 2

| Comparative Example | 1 | 2 | 3 | 4 |
|---|---|---|---|---|
| Porous Adsorbent | Activated carbon | Activated carbon | — | Activated carbon |
| Amount of adsorbent (g) | 0.4 | — | — | less than 0.05 |
| Binder | — | — | Mixture of crystalline | Polyvinyl alcohol |
| | | | cellulose (98%) and | |
| | | | silica (2%) | |
| Amount of binder (g) | — | — | 0.4 | more than 0.95 |
| Additive | — | — | — | — |
| Compression tableting | over 20 | — | 4 to 5 | Not conducted |
| pressure (kN) | | | | |
| Weight of tablet (g) | 0.4 | | 0.4 | 0.1 |
| Diameter of tablet (mm) | 10 | | 10 | 10 |
| Hardness (N) | Tablet was not | — | over 20 | 6 |
| | formed | | | |
| Distributed over soil | — | Powder flew | No change of shape | Surface geled |
| Disintegration time in soil | — | — | No change | over 1 day |
| Distributed on water | — | Floated on water | Tablet sank in water | Tablet floated and |
| | | surface | | did not disintegrate |
| Disintegration time in water | — | — | over 600 sec. | over 600 sec. |

### Example 7

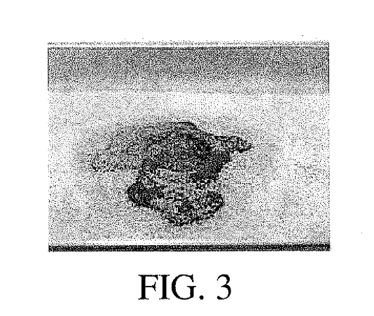

A tablet with a diameter of 20 mm was prepared using the same porous adsorbent and binder as those in Example 1. The tablet was placed on the soil, over which 25 mL, of water was dropped using a pipette over 10 seconds. This was repeated thrice. FIG. 3 is a photograph showing the state of the tablet 60 seconds after the start of dropping of water. It was found that the tablet disintegrated within 30 seconds by application of a small amount of water and the disintegrated particles flew into the interstices of the soil as shown in FIG. 3. The soil and the adsorbent were found to well contact with each other. Thus the tablet was found to be usable in arid zone soil or for agriculture using a small amount of water and to be high in safety.

### Example 8

Three types of tablets, Tablets A, Tablets B and Tablets C, each having a weight of 0.4 g were prepared using the same activated carbon, crystalline cellulose and silicon dioxide as used in Example 2. Tablets A were prepared by compression tableting a mixture of 2 parts of activated carbon, 18 parts of silicon dioxide and 20 parts of crystalline cellulose. Tablets B were prepared by compression tableting a mixture of 10 parts of activated carbon, 10 parts of silicon dioxide and 20 parts of crystalline cellulose. Further, Tablets C were prepared by compression tableting a mixture of 18 parts of activated carbon, 2 parts of silicon dioxide and 20 parts of crystalline cellulose. Each of the three types of tablets was subjected to a test of adsorbing persistent organic pollutants (POPs) contained in a bottom sediment (sludge) of a river.

### Sampling Method:

From air-dried sludge of a river, 20 g of a soil sample were weighed and placed in a vessel. After addition of 600 mL of pure water, the vessel was closed and shaken for 1 week. The resulting mixture was allowed to quiescently stand, from which 200 mL of the supernatant was collected to obtain sample 1W. The obtained sample 1W was analyzed for POPs. The remainder of the mixture in the vessel was added with 200 mL of pure water, to which 7 pieces of Tablets A were added. The vessel was then shaken for 1 week. The mixture in the vessel was allowed to quiescently stand, from which 200 mL of the supernatant was sampled. The obtained sample 2W was analyzed for POPs. The remainder of the mixture in the vessel was added with 200 mL of pure water, shaken for 1 week and allowed to quiescently stand, from which 200 mL of the supernatant was sampled. The obtained sample 3W was analyzed for POPs. The remainder of the mixture in the vessel was added with 200 mL of pure water, shaken for 1 week and allowed to quiescently stand, from which 200 mL of the supernatant was sampled. The obtained sample 4W was analyzed for POPs. The above procedures were repeated in the same manner as described except for using Tablets B and C in place of Tablets A. For the purpose of comparison, the above procedures were repeated in the same manner as described except that no tablets were added.

### Measurement Conditions:

Each of the 200 mL supernatant (samples 1W to 4W) sampled every week was filtered through a glass filter and added to a separation funnel containing 100 mL of hexane. This was shaken for 30 minutes. Such liquid-liquid extraction was conducted 3 times. The hexane layer was concentrated and purified on a Florisil column with 100 mL of 25% dichloromethane and 75% hexane and then on an ENVI-carb SPE column with 10 mL of hexane. Analysis of POPs was carried out using a high resolution GC/MS under the following conditions:

GC oven temperature: 120° C. (0.5 min)-10° C./min-180° C. (0 min)-4° C./min-210° C. (0 min)-10° C./min-300° C. (10 min);
GC column: ENV-8MS (0.25 mm inside diameter×30 m);
Injection method: splitless;
Injection amount: 1 μL;
Ionization method: EI+;
Resolution: 10000 or higher (10% valley);
Analysis method: SIM method (accurate mass (m/z) was measured in the low and high mass regions (as shown below) separately so as to enable simultaneous measurement of all components. Each compound was measured by an internal standard method using 13C;
High Mass Region (m/z):
Trans, cis-heptachloroepoxide 352.8442, 354.8413, 13C,
Trans, cis-heptachloroepoxide 362.8778, 364.8748,
Trans, cis-chlorodene 372.826, 374.823, 13C,

### Cis-chlorodene 382.8595, 384.8566,

### Oxychlorodene 386.8052, 388.8023, 13C,

### Oxychlorodene 396.8388, 398.8358,

Trans, cis-nonachlor 406.787, 408.784, 13C,
Trans, cis-nonachlor 416.8205, 418.8176;
Low Mass Region (m/z):

### Hexachlorocyclohexane (HCH) 216.9145, 218.9116, 13C,

### Hexachlorocyclohexane 222.9347, 224.9317,

### Dichlorodiphenyltrichioroothane (DDT), Dichlorodiphenyldichloroethane (DDD) 235.0081, 237.0052, 13C DDT, DDD 247.0484, 249.0454,

### Dichlorodiphenyldichloroethylene (DDE) 246.0003, 247.9974, 13C

### DDE 258.0406, 260.0473,

### Aldrin, Dieldrin, Endrin 262.857, 264.854, 13C

### Aldrin, Dieldrin, Endrin 269.8804, 271.8775,

### Mirex, Heptachloro 271.8102, 273.8072, 13C,

### Mirex, Heptachloro 276.8269, 278.824,

### Hexachlorobenzene (HCB) 283.8102, 285.8072, 13C

### Hexachlorobenzene 289.8303, 291.8273.

### Test Results:

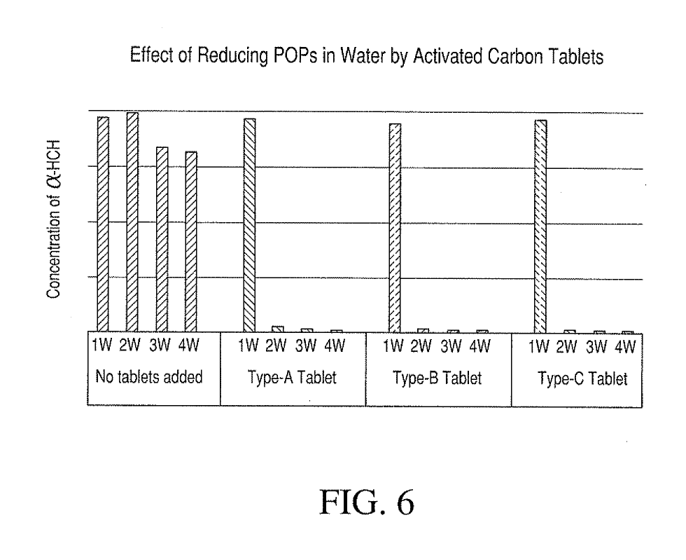

When the tablets were not added, the concentration of the POPs leached from the sludge into water was high in any of the supernatant samples 1W to 4W. In contrast, when Tablets A to C were added, the POPs contained in the sludge were found to be effectively adsorbed by the tablets within 1 week after the addition of the tablets, although there were certain time differences in adsorption rate depending upon the kinds thereof. An example of the adsorption behavior of α-HCH (α-hexachlorocyclohexane) as a typical example of POPs is shown by way of a graph in FIG. 6, from which it is evident that α-HCH was remarkably decreased within 1 week. Similar results were also obtained in the case of other POPs exemplified above. In FIG. 6, the ordinate represents a concentration ratio of α-HCH relative to the maximum concentration of α-HCH leached into water from the sediment.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all the changes which come within the meaning and range of equivalency of the claims are therefore intended to be embraced therein.

What is claimed is:

1. A hazardous substance adsorbing tablet prepared by direct compression of a dry powder mixture, said powder mixture comprising:
of a porous particulate adsorbent having an average particle diameter of 100 μm or less and containing 200 mesh pass particles in an amount of more than 80% by weight, and
a particulate binder having an apparent specific gravity of at least 1.

2. The tablet according to claim 1, wherein the tablet disintegrates within 10 minutes when added into water at 4° C.

3. The tablet according to claim 1, wherein the porous particulate adsorbent is at least one substance selected from the group consisting of calcite, naturally occurring zeolite, artificial zeolite, activated clay, diatomaceous earth, granite pegmatite, ilmenite and activated carbon having a specific surface area of 800 m²/g or more.

4. The tablet according to claim 1, wherein the porous particulate adsorbent and the particulate binder are present in amounts of 5 to 90% by weight and 10 to 95% by weight, respectively, wherein the total weight of the adsorbent and the binder is 100% by weight.

5. The tablet according to claim 1, wherein the porous particulate adsorbent has an average particle diameter of at least 1 μm

6. The tablet according to claim 1, wherein the binder comprises at least one substance selected from the group consisting of crystalline cellulose, carboxymethyl cellulose, hydroxypropyl cellulose, starch, lactose, silicon dioxide, mannitol and anhydrous calcium hydrogen phosphate.

7. The tablet according to claim 1, wherein the binder having an apparent specific gravity of at least 1 comprises 1 to 130 parts by weight of silicon dioxide having an apparent specific gravity greater than 1 and 100 parts by weight of crystalline cellulose.

8. The tablet according to claim 7, wherein the crystalline cellulose has an average particle diameter 50 to 250 μm and silicon dioxide has an average particle diameter of 1 to 30 μm.

9. The tablet according to claim 1, further comprising at least one particulate adsorbing substance selected from the group consisting of inorganic salts which can form sparingly soluble heavy metal salts with heavy metal ions, inorganic sulfides which can form sparingly soluble heavy metal compounds with heavy metal ions, chelating agents, pH controlling agents, polyamino acids, polyacrylic acids and chelating agents.

10. The tablet according to claim 1, further comprising at least one additive selected from the group consisting of oxidizing agents, reducing agents, microbial decomposing bacteria, slow oxygen-releasing agents, nutrients for growing bacteria, fertilizers, agricultural chemicals, and antibacterial antiseptic agents.

11. The tablet according to claim 8, wherein the porous particulate adsorbent is at least one substance selected from the group consisting of calcite, naturally occurring zeolite, artificial zeolite, activated clay, diatomaceous earth, granite pegmatite, ilmenite and activated carbon having a specific surface area of 800 m²/g or more.

12. A method for treating a polluted environment, comprising applying the tablet as recited in claim 1 to the polluted environment.

13. The method according to claim 12, wherein the polluted environment is water or soil.
